(19) United States
(12) Patent Application Publication
(10) Pub. No.: US 2025/0109114 A1
(43) Pub. Date: Apr. 3, 2025

(54) A SYNTHESIS OF POTASSIUM 5,7-DINITRO-[2,1,3]-BENZOXADIAZOL-4-OLATE-3-OXIDE

(71) Applicants: BRAJA SUNDAR PRADHAN, Hyderabad (IN); NEHA MEHTA, Picatinny Arsenal, NJ (US); KEYUR PATEL, Picatinny Arsenal, NJ (US)

(72) Inventors: BRAJA SUNDAR PRADHAN, Hyderabad (IN); NEHA MEHTA, Picatinny Arsenal, NJ (US); KEYUR PATEL, Picatinny Arsenal, NJ (US)

(21) Appl. No.: 18/374,663

(22) Filed: Sep. 29, 2023

(51) Int. Cl. C07D 271/12

(52) U.S. Cl. CPC C07D 271/12

(57) ABSTRACT

Embodiments of this disclosure describe a method for synthesizing potassium 5,7-dinitro-[2,1,3]-benzoxadiazol-4-olate-3-oxide (benzofuroxan derivative). In the method, a nucleophilic displacement reaction was conducted between sodium azide and 3-chloro-2,4,6-trinitrophenol. The reaction was performed at a controlled temperature using a suitable reaction medium consisting of water and methanol. After the completion of the reaction, the mixture was allowed to cool down to room temperature. On further cooling, a solid precipitated out of the reaction mixture. The solid is filtered and air-dried to give the sodium analogue of the benzofuroxa derivative. The analogue is a crucial precursor of the benzofuroxan derivative. The sodium cation is subsequently replaced with potassium by carrying the precursor through a series of operations. First, it was treated with a methanolic hydrochloric acid. The treatment removed the sodium cation and introduced a proton in its place. Sodium chloride thus was filtration off. The filtrate was concentrated under reduced pressure to obtain the penultimate precursor of the benzofuroxan derivative. Finally, the precursor was treated with potassium tert-butoxide to yield a benzofuroxan derivative in good yield and purity.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter generally relates to the field of furoxan chemistry. More particularly, the present invention relates to a novel, practical method of the preparation of potassium 5,7-dinitro-[2,1,3]-benzoxadiazol-4-olate-3-oxide, which is also alternatively referred to as 4,6-dinitro-7-hydroxybenzofuroxan, potassium (benzofuroxan derivative).

### BACKGROUND

In the field of Hamiltonian materials, using lead-based compounds, such as lead styphnate, has raised concerns due to their potential environmental and health risks. Consequently, there is a growing demand for alternative, environmentally friendly substances with desirable performance characteristics. One alternative is potassium 5,7-dinitro-[2,1,3]-benzoxadiazol-4-olate-3-oxide (benzofuroxan derivative), a promising substitute for lead-based materials.

However, the existing synthetic methods for the benzofuroxan derivative face several challenges that hinder their practicality for commercial production. One significant issue is the availability and cost of specific reagents, particularly potassium azide, which is crucial in the current synthesis process. The limited accessibility and high expense of potassium azide make it economically impractical for the widespread manufacture of the benzofuroxan derivative.

Furthermore, the current synthetic route necessitates elevated temperature to form the furoxan ring in the benzofuroxan derivative. This requirement raises safety concerns, as subjecting such compounds to high temperatures can pose risks during the commercial production. Therefore, there is an apparent necessity for developing an alternative synthetic method that is not only safer but also addresses the limitations of the current approaches.

In light of these considerations, exploring a novel, alternate synthetic process that can overcome the drawbacks associated with the existing methods is crucial. This process should offer an environmentally friendly approach that employs readily available and cost-effective reagents, and ensures commercially safe production of the benzofuroxan derivative.

Given the challenges above, there is a clear need to develop an alternative, safer process that fulfills these requirements.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards synthesizing 5,7-dinitro-[2,1,3]-benzoxadiazol-4-olate-3-oxide (benzofuroxan derivative).

The present disclosure aims to introduce a new, practical, and efficient method for synthesizing the benzofuroxan derivative.

Another objective of the present disclosure is to overcome the drawbacks of the existing methods, such as the high cost and limited availability of reagents, particularly potassium azide, and the safety concerns associated with high-temperature reaction condition.

Another objective of the present disclosure is to utilize readily accessible and cost-effective reagents in the synthesis.

Another objective of the present disclosure is to improve the availability and affordability of the benzofuroxan derivative, enabling its widespread use as a viable alternative to lead-based compounds.

Another objective of the present disclosure is to develop a synthetic process that minimizes the environmental impact and reduces the reliance on hazardous substances, such as lead-based compounds.

Another objective of the present disclosure is to establish a synthetic method suitable for commercially producing the benzofuroxan derivative.

Another objective of the present disclosure is to facilitate manufacturing the benzofuroxan derivative in quantities that meet the demands of various applications within the Hamiltonian materials field.

Another exemplary embodiment of the present disclosure may involve developing a method that utilizes sodium azide as the azide donor, which is readily available on a commercial scale and offers cost advantages.

Another exemplary embodiment of the present disclosure may involve implementing a nucleophilic displacement reaction between sodium azide and 3-chloro-2,4,6-trinitrophenol, producing a precursor compound in good yield and purity.

Another exemplary embodiment of the present disclosure may involve designing a solvent system comprising water and methanol that ensures the homogeneity of the reaction mixture, facilitating the formation of the furoxan ring in one-step.

Another exemplary embodiment of the present disclosure may involve eliminating the need to isolate the azido intermediate and applying high temperature to create the desired furoxan ring, thus enhancing the safety of the process.

Another exemplary embodiment of the present disclosure may involve precipitating the precursor compound directly from the reaction mixture, minimizing the need for additional purification steps.

Another exemplary embodiment of the present disclosure may involve developing a procedure for the conversion of the sodium analogue (sodium 5,7-dinitro-[2,1,3]-benzoxadiazol-4-olate-3-oxide) to the desired potassium compound through the treatment with an acid, preferably methanolic hydrochloric acid and a potassium alkoxide, preferably potassium tert-butoxide.

Another exemplary embodiment of the present disclosure may involve achieving the final synthesis of the benzofuroxan derivative in good yield and purity, rendering the process suitable for various applications within the field of Hamiltonian materials.

These exemplary embodiments demonstrate the innovative aspects of the process, highlighting the utilization of sodium azide as a cost-effective reagent, the specific solvent system as an efficient protocol for the furoxan ring formation, and the sequential treatments to achieve the final synthesis of the benzofuroxan derivative. Each embodiment showcases the practicality, efficiency, and potential scalability of the novel synthetic method, providing the benzofuroxan derivative in high purity for diverse applications within the field of Hamiltonian materials.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

Following the non-limiting exemplary embodiment of the present disclosure, preparing potassium 5,7-dinitro-[2,1,3]-benzoxadiazol-4-olate-3-oxide (benzofuroxan derivative), may involve a series of interconnected steps.

In the first step, a nucleophilic displacement reaction may be conducted, wherein sodium azide, a potentially cost-effective and readily available azide donor, may be treated with 3-chloro-2,4,6-trinitrophenol in a suitable reaction medium. The reaction may be carried out at a controlled temperature of 80-85° C. To potentially ensure homogeneity of the reaction mixture, the suitable reaction medium comprises a solvent system consisting of water and methanol in a particular ratio. This solvent system was carefully selected based on its ability to provide homogeneity to the reaction mixture. The homogeneity of the mixture plays a crucial role in facilitating the formation of the furoxan ring in a single step, distinguishing it from conventional processes. The advantage of this approach is that it eliminates the need to isolate the azido intermediate, specifically 3-azido-2,4,6-trinitrophenoxide, and avoids the requirement of subjecting it to high temperature in refluxing diethyl carbonate for the cyclization. The nucleophilic displacement reaction may occur between the azide and the chloro-substituted compound, potentially forming a precursor compound in a potentially excellent yield and purity.

In the subsequent step, once the reaction is potentially complete, the reaction mixture may be cooled to a temperature range of 0-5° C. The precursor compound may precipitate out of the mixture at this temperature. The solid precursor may then be separated from the reaction mixture by filtration. Following the filtration, the precursor may be air-dried. Notably, the precursor obtained may not require further purification and can proceed directly to the subsequent steps.

Next, the precursor compound may have a molecular architecture identical to the benzofuroxan derivative but contain a sodium cation instead of a potassium cation. To replace the sodium cation with potassium, the precursor, known as sodium 5,7-dinitro-[2,1,3]-benzoxadiazol-4-olate-3-oxide, may be treated with an acid, preferably methanolic hydrochloric acid. This treatment may facilitate the potential removal of the sodium cation, resulting in the formation of sodium chloride as a byproduct. The sodium chloride may be separated from the reaction mixture via filtration. The resulting filtrate, potentially containing the desired intermediate, may then be concentrated under reduced pressure. This concentration step may lead to the formation of the penultimate precursor, namely, 5,7-dinitro-[2,1,3]-benzoxadiazol-4-ol-3-oxide required for the synthesis of the benzofuroxan derivative.

In the final step, the penultimate intermediate compound,5,7-dinitro-[2,1,3]-benzoxadiazol-4-ol-3-oxide, may be treated with potassium tert-butoxide in methanol. This treatment may allow for the final conversion and synthesis of the benzofuroxan derivative, representing the primary objective of the process. The reaction may proceed smoothly, producing the benzofuroxan derivative in good yield and purity. The synthesized benzofuroxan derivative can then be further processed and potentially find applications in various fields within the domain of Hamiltonian materials.

This exemplary embodiment showcases a potential method for preparing the benzofuroxan derivative, starting with a nucleophilic displacement reaction and progressing through the precipitation of the precursor, the sodium analogue, and its conversion to the penultimate intermediate, and, finally, the transformation of the intermediate to the desired potassium compound, the benzofuroxan derivative. Each step in the process may contribute to the efficient synthesis and purification of the benzofuroxan derivative, potentially ensuring the production of a high-quality compound suitable for various applications.

### WORKING EXAMPLE: 1

In another possible embodiment, sodium azide (19.1 g) may be added in divided batches to a solution of 3-chloro-2,4,6-trinitrophenol in methanol at 50° C. over a period of 5-10 minutes. Subsequently, water (500 ml) may be added to the mixture at this temperature, and the mixture may be heated to 80-85° C. On completion of the reaction as indicated by thin layer chromatography (tlc), the mixture may be allowed to cool down to room temperature. Upon further cooling to a range of 0-5° C., a potentially yellow-colored solid may precipitate out of the mixture. The solid may then be filtered, washed with water and air dried to obtain sodium 5,7-dinitro-[2,1,3]-benzoxadiazol-4-olate-3-oxide (NaDNP) with a potential yield of 54 g. The resulting material may be carried over to the following step without further purification.

### WORKING EXAMPLE: 2

In another potential scenario, sodium 5,7-dinitro-[2,1,3]-benzoxadiazol-4-olate-3-oxide (54 g) may be stirred in methanolic hydrochloric acid at room temperature until the precipitation of sodium chloride is deemed complete. The resulting precipitate may be filtered off, and the resulting filtrate may be concentrated under reduced pressure. This process may lead to the formation of the hydroxy compound, 5,7-dinitro-[2,1,3]-benzoxadiazol-4-ol-3-oxide, potentially yielding 37 g of the desired product.

### WORKING EXAMPLE: 3

In a potential scenario, a solution of potassium tert-butoxide (16.4 g) in methanol (55 ml) may be gradually added drop by drop to a solution of 5,7-dinitro-[2,1,3]-benzoxadiazol-4-ol-3-oxide (37 g) in methanol at room temperature over a period of 0.5 hour. The resulting mixture may be stirred at this temperature until the precipitation of a potentially yellow-colored solid is considered complete. The solid may then be filtered, washed with isopropyl alcohol, and dried to obtain the benzofuroxan derivative with a potential yield of 34 g.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A method of synthesis of potassium 5,7-dinitro-[2,1,3]-benzoxadiazol-4-olate-3-oxide (benzofuroxan derivative), the method comprising:
a. conducting a nucleophilic displacement reaction between sodium azide and 3-chloro-2,4,6-trinitrophenol in a suitable reaction medium at a controlled temperature of 80-85° C., thereby forming a precursor compound;
b. cooling the reaction mixture to a temperature range of 0-5° C. to precipitate the precursor compound;
c. separating the solid precursor compound from the reaction mixture by filtration;
d. air-drying the precursor compound without further purification;
e. treating the precursor compound with methanolic hydrochloric acid to remove the sodium cation and generate sodium chloride as a byproduct;
f. filtering out the sodium chloride and concentrating the resulting filtrate under reduced pressure, forming the penultimate intermediate compound required for synthesizing the benzofuroxan derivative; and
g. The synthesis involves subjecting the penultimate intermediate compound to a treatment with a potassium alkoxide, preferably potassium tert-butoxide in methanol, to convert it into potassium 5,7-dinitro-[2,1,3]-benzoxadiazol-4-olate-3-oxide (benzofuroxan derivative) with good yield and purity.

2. The method of claim 1, wherein the suitable reaction medium comprises a solvent system consisting of water and methanol in a specific ratio to ensure homogeneity of the reaction mixture, facilitating a one-step formation of the furoxan ring in one step.

3. The method of claims 1, wherein the precursor compound is sodium 5,7-dinitro-[2,1,3]-benzoxadiazol-4-olate-3-oxide.

4. The method of claim 1, wherein the precursor compound obtained in the nucleophilic displacement reaction is directly utilized in subsequent steps without isolation or purification.

5. The method of claim 1, wherein the precursor compound is directly treated with methanolic hydrochloric acid without isolation or purification.

6. The method of claim 1, wherein the penultimate intermediate compound is 5,7-dinitro-[2,1,3]-benzoxadiazol-4-ol-3-oxide.

7. The method of claim 1, wherein the penultimate intermediate compound obtained after removing sodium chloride is directly treated with a potassium alkoxide, preferably potassium tert-butoxide, in methanol without isolation or purification.

8. The method of claim 1, wherein the treatment with methanolic hydrochloric acid comprises stirring the precursor compound in the presence of methanolic hydrochloric acid at room temperature until the precipitation of sodium chloride is complete.

9. The method of claim 1, wherein the filtrate obtained after removing sodium chloride is concentrated under reduced pressure using a specific technique.

10. The method of claim 1, wherein the treatment with potassium tert-butoxide in methanol is performed by gradually adding a potassium tert-butoxide in methanol to the penultimate intermediate compound solution at room temperature.
